(19) United States
(12) Patent Application Publication
(10) Pub. No.: US 2012/0046265 A1
(43) Pub. Date: Feb. 23, 2012

(54) USE OF ERGOSTA-7,22-DIEN-3beta-OL IN THE TREATMENT OF CANCER

(75) Inventor: Hei Ling Helen CHAN, Hong Kong (HK)

(73) Assignee: VITA GREEN BIOTECHNOLOGY CO. LIMITED, Hong Kong (HK)

(21) Appl. No.: 13/283,619

(22) Filed: Oct. 28, 2011

Related U.S. Application Data
(62) Division of application No. 12/406,112, filed on Mar. 18, 2009.
(60) Provisional application No. 61/040,137, filed on Mar. 27, 2008.

(51) Int. Cl. A61K 31/575; A61P 35/02; A61P 35/00

(52) U.S. Cl. 514/182

(57) ABSTRACT

A use of an isolated and purified form of ergosta-7,22-dien-3β-ol in the treatment of cancer is provided.

### CROSS-REFERENCE TO RELATED APPLICATION

This application is a divisional application of patent application Ser. No. 12/406,112 filed on 18 Mar. 2009, which claims benefit under 35 U.S.C. §119(e) of U.S. Provisional Application having Ser. No. 61/040,137 filed Mar. 27, 2008, which is hereby incorporated by reference herein in its entirety.

### FIELD OF INVENTION

This invention relates to an isolated and purified compound of ergosta-7,22-dien-3β-ol, and its use as a marker for identifying Coriolus versicolor (Yunzhi) The present invention also relates to methods of identifying Coriolus versicolor in samples using identification marker.

### BACKGROUND OF INVENTION

Coriolus versicolor (Yunzhi) has been commonly used as a medicinal herb in Traditional Chinese Medicine.

Yunzhi, also known as Polyporus versicolor, Trametes versicolor, Turkey Tail, or cloud mushroom, is an edible fungus that belongs to the Basidiomycetes class and Polyporaceae family. It has been investigated in laboratories worldwide and studies have demonstrated that it possesses immune modulating and boosting function, and other significant effects. Since these discoveries, there has been an increasing demand for the supply for this herb. Although polysaccharide ingredients have been reported, none of these ingredients has been successfully identified as fingerprints.

### SUMMARY OF INVENTION

In the light of the foregoing background, it is an object of the present invention to provide markers for identifying Coriolus versicolor (Yunzhi) and methods of identifying same.

Accordingly, the present invention, in one aspect, is an isolated and substantially purified ergosta-7,22-dien-3β-ol with the following formula (I):

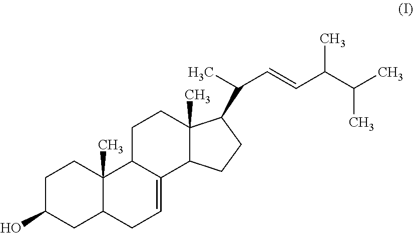

According to another aspect of the present invention, the use of ergosta-7,22-dien-3β-ol represented by formula (I) as a marker for identifying Coriolus versicolor (Yunzhi) is provided.

In a further aspect of the present invention, a method of quantifying Coriolus versicolor in a sample is provided which comprises first extracting components from Coriolus versicolor to form a sample extract. The sample extract is then separated using a separation technique to obtain a separation result, followed by identifying ergosta-7,22-dien-3β-ol (represented by formula (I)) from the separation result. The quality of the sample extract is subsequently determined according to pre-determined levels of ergosta-7,22-dien-3β-ol in the sample extract.

In a non-limiting embodiment of the above method, chromatography was used as the separation technique. In another non-limiting embodiment, the chromatography used is gas chromatography (GC) with the running conditions as shown in Table 4 below. In yet another non-limiting embodiment, the chromatography employed is high performance liquid chromatography (HPLC) with the running conditions as shown in Table 3 below.

In yet another aspect of the present invention, a composition comprising ergosta-7,22-dien-3β-ol is used to be administrated for the treatment of cancer and other related diseases or disorders. In a non-limiting embodiment, the type of cancer includes but not limited to leukemia. In another non-limiting embodiment, the ergosta-7,22-dien-3β-ol is admixed with other herb(s) and/or pharmaceutically acceptable carrier(s).

In another aspect of the present invention, a method of treating cancer and other related diseases or disorders comprising administrating an effective dose of a composition containing ergosta-7,22-dien-3β-ol is provided. In a non-limiting embodiment, the type of cancer includes but not limited to leukemia. In another non-limiting embodiment, ergosta-7,22-dien-3β-ol is admixed with other herb(s) and/or pharmaceutically acceptable carrier(s).

The use of the marker according to this invention may provide a fast and accurate method for qualifying and quantifying the medicinal herb, as compared to the traditional way of conducting a long series of tests and studies to reach the same results.

Furthermore, the use of marker may find it useful and efficient in identifying and pinpointing the medicinal herb of interest among other ingredients within an herbal mixture.

### BRIEF DESCRIPTION OF FIGURES

FIG. 1 is an HPLC spectrum for a sample of impure ergosta-7,22-dien-3β-ol prepared according to one embodiment of the present invention.

FIG. 2 is an HPLC spectrum for a sample of substantially purified ergosta-7,22-dien-3β-ol prepared according to the same embodiment of the present invention.

FIG. 3 is an HPLC spectrum for a sample of Yunzhi plant material according to the same embodiment of the present invention.

FIG. 4 is a GC spectrum for a sample of impure ergosta-7,22-dien-3β-ol prepared according to one embodiment of the present invention.

FIG. 5 is a GC spectrum for a sample of substantially purified ergosta-7,22-dien-3β-ol prepared according to the same embodiment of the present invention.

FIG. 6 is a GC spectrum for a sample of Yunzhi plant material according to the same embodiment of the present invention.

### DETAILED DESCRIPTION OF THE PREFERRED EMBODIMENTS

As used herein and in the claims, “comprising” means including the following elements but not excluding others. When interpreting each statement in this specification that includes the term “comprising”, features other than that or those prefaced by the term may also be present. Related terms such as “comprise” and “comprises” are to be interpreted in the same manner.

The present invention provides an isolated and substantially purified ergosta-7,22-dien-3β-ol as represented by formula (I) above. In one embodiment, the melting point of the ergosta-7,22-dien-3β-ol solid prepared is 169 to 172° C.

In the course of making the present invention, it has been discovered that purified ergosta-7,22-dien-3β-ol also possesses similar biological activities (e.g. anti-leukemia effects) as Coriolus versicolor. As such, not only there is a direct relationship between the purity of ergosta-7,22-dien-3β-ol and its quantification as a marker for Coriolus versicolor, but the concentration of ergosta-7,22-dien-3β-ol is also an indication of the effectiveness of a preparation containing ergosta-7,22-dien-3β-ol.

The present invention also pertains to a method of quantifying Coriolus versicolor (Yunzhi) in a sample containing unknown amount of Yunzhi. This method comprises (a) extracting components from Yunzhi in the sample to form a sample extract; (b) separating the sample extract using a separation technique to obtain a separation result; (c) identifying ergosta-7,22-dien-3β-ol from the separation result; and (d) determining the quality of the sample extract according to pre-determined levels of ergosta-7,22-dien-3β-ol in the sample extract.

In a non-limiting embodiment of the above method, chromatography was used as the separation technique. In another non-limiting embodiment, the chromatography used is gas chromatography (GC) with the running conditions as shown in Table 4 below. In yet another embodiment, the chromatography employed is high performance liquid chromatography (HPLC) with the running conditions as shown in Table 3 below.

In one non-limiting embodiment, a sample containing unknown amount of Yunzhi was ultrasonically extracted with chloroform:methanol (2:1) in a volumetric flask for 20 minutes. After drying, chloroform and an internal standard solution (20 mg of 5α-cholestane in 2 ml of chloroform) were added to this extract. The sample extract thus formed was filtered through 0.45 μm filter into vials as specimen for GC analysis. From the GC spectrum generated, the peak of ergosta-7,22-dien-3β-ol was identified. The quality of Yunzhi present in the sample was finally determined according to the pre-determined levels of ergosta-7,22-dien-3β-ol present in the sample extract.

In another non-limiting embodiment, a sample containing unknown amount of Yunzhi was ultrasonically extracted with an aqueous mixture of THF-Tween 80 (in the ratio of 1:1, (THF/Tween 80):water) in a volumetric flask for 10 minutes. Then, acetonitrile was added to fill up to the mark and the resulting solution was sonicated for another 5 minutes. The sample extract thus formed was filtered through 0.45 μm filter into vials as specimen for HPLC analysis. From the HPLC spectrum generated, the peak of ergosta-7,22-dien-3β-ol was identified. The quality of Yunzhi present in the sample was finally determined according to the pre-determined levels of ergosta-7,22-dien-3β-ol present in the sample extract.

In a non-limiting embodiment of the aforesaid quantifying method, purified Yunzhi powder was quantified by first extracting ergosta-7,22-dien-3β-ol from a known amount of the purified Yunzhi powders. Upon purification, the weight of the purified ergosta-7,22-dien-3β-ol solids was measured to determine the concentration of ergosta-7,22-dien-3β-ol in the purified Yunzhi powders.

The present invention is further defined by the following examples, which are not intended to limit the present invention. Reasonable variations, such as those understood by reasonable artisans, can be made without departing from the scope of the present invention.

### Example 1

Preparation of Purified Ergosta-7,22-Dien-3β-ol Powder from Purified Yunzhi Powder

1.1 Crude Fluid Extract Preparation from Yunzhi Pure Powder

Purified Yunzhi powder was first obtained by extracting and concentrating Yunzhi raw herbs according to the purification method disclosed in U.S. patent application Ser. No. 11/269,294 filed on Nov. 8, 2005.

For every 1.5 kg of purified Yunzhi powder used, 6 to 7 L of chloroform was employed as an extraction medium inside a flask connecting to the Liebig condenser. This mixture was heated at 70° C. (under reflux) for 3 to 3.5 hours. Upon cooling, the heated mixture was filtered through the Buchner funnel and the solvent in the filtrate was removed under reduced pressure. The resulting filtrate extract was saved while the solid residue was extracted according to the aforesaid procedure for at least twice. At the end, all the filtrate extracts were combined to obtain the crude fluid extract.

1.2 Column Separation of the Fluid Crude Extract

A silica gel sample was prepared by first mixing about 20 to 25 g of the crude fluid extract from step 1.1 with 25 to 30 g of silica gel (70-230 mesh), followed by allowing the solvent within the silica gel sample to be completely evaporated out. The separation column, in one embodiment, was prepared by packing 500 to 550 g of silica gel (230-400 mesh) pre-mixed with petroleum ether into a column (5×60 cm). The mobile phase of the column was prepared as follows (Table 1):

TABLE 1

| Polarity (% ethyl acetate) | Volume per each addition (mL) |
|---|---|
| 5 | 3000 |
| 8 | 2000 |
| 11 | 2000 |
| 14 | 2500 |
| 20 | 2500 |

In another embodiment, the separation column was prepared by packing 500 g of silica gel (230-400 mesh) mixed with petroleum ether into a column (8×30 cm). The mobile phase was provided according to Table 2 below:

TABLE 2

| Polarity (% ethyl acetate) | Volume per each addition (mL) |
|---|---|
| 5 | 2000 |
| 8 | 1500 |
| 11 | 2500 |
| 14 | 1500 |
| 18 | 1500 |
| 21 | 2500 |

Afterwards, the silica gel sample was loaded into the column and fractions were collected until the first yellow band moved near to the bottom of the column. The volume of each fraction collected ranged approximately 240 to 260 mL.

1.3 Identifying Fractions Collected from Column Separation Using Thin-Layer Chromatography

Fractions collected from column separation as described in step 1.2 were analyzed using thin-layer chromatography (TLC). In this step, the fractions and control were both dotted on a thin plate of silica gel 60 with fluorescent dye and developed using 20% ethyl acetate in hexane. The spots on the developed plates were then stained with vanillin stain or visualized using UV irradiation at 254 nm and 365 nm

Fractions having the same retardation factor (Rf) value with reference were combined according to the TLC results. In one embodiment, fractions having the Rf value of 0.2, which corresponds to ergosta-7,22-dien-3β-ol, were combined and obtained as the crude product.

1.4 Re-Crystallizing the Crude Product for Preparation of Purified Ergosta-7,22-dien-3β-ol

The crude product from step 1.3 above was dissolved in chloroform and methanol.

For every 1 g of products used, about 20 mL of chloroform and about 5 mL of methanol were employed. Upon precipitation, an extra volume of about 3 mL of methanol was added and the mixture was allowed to stand for 1 to 2 days for complete re-crystallization. Finally, purified crystals of ergosta-7,22-dien-3β-ol were obtained. In one embodiment, 0.5 g of the purified crystals of ergosta-7,22-dien-3β-ol was obtained as the final product.

1.5 Evaluation of the Purity of the Final Product

The crystals of ergosta-7,22-dien-3β-ol from step 1.4 were determined to be pure based on the melting point analysis in which a result of 169-172° C. was recorded. Other characterization techniques were used to determine the structure of this compound. Further, gas chromatography (GC) and/or high performance liquid chromatography (HPLC) analysis was also conducted for evaluating the purity of the product.

1.5.1 High Performance Liquid Chromatography (HPLC)

1.5.1.1 Preparation of Specimen

About 500 mg of the sample tested (i.e. the purified extract of ergosta-7,22-dien-3β-ol obtained from step 1.4) was dissolved with 40 mL of an aqueous mixture of THF-Tween 80 (in the ratio of 1:1, (THF/Tween 80): water) in a 50 mL volumetric flask. Then, acetonitrile was added to fill up to the mark. The solution thus formed was filtered through 0.45 μm filter into vials as specimen for HPLC detection.

1.5.1.2 Preparation of Samples of Impure ergosta-7,22-dien-3β-ol

About 500 mg of the ergosta-7,22-dien-3β-ol crude product obtained from step 1.3 above was dissolved with 40 mL of an aqueous mixture of THF-Tween 80 (in the ratio of 1:1, (THF/Tween 80): water) in a 50 mL volumetric flask. Then, acetonitrile was added to fill up to the mark. The solution thus formed was filtered through 0.45 μm filter into vials as specimen for HPLC detection.

1.5.1.3 HPLC System

The entire HPLC system and all its mechanical parts were purchased from Waters Corporation, Mass., USA. The instruments used included Waters 2695 Separation Module and Waters 2996 Photodiode Array Detector.

1.5.1.4 Running Conditions

The running conditions for this HPLC analysis are shown in Table 3 below.

TABLE 3

| Column: | C18 | |
|---|---|---|
| Column temperature: | 30° | C. |
| Wavelength: | 282 | nm |
| Flow rate: | 1.0 | mL/min |
| Injection volume: | 20 | μL |

| | |
|---|---|
| Mobile phase: | 98.5% acetonitrile - 1.5% water |

1.5.1.5 Results and Analysis

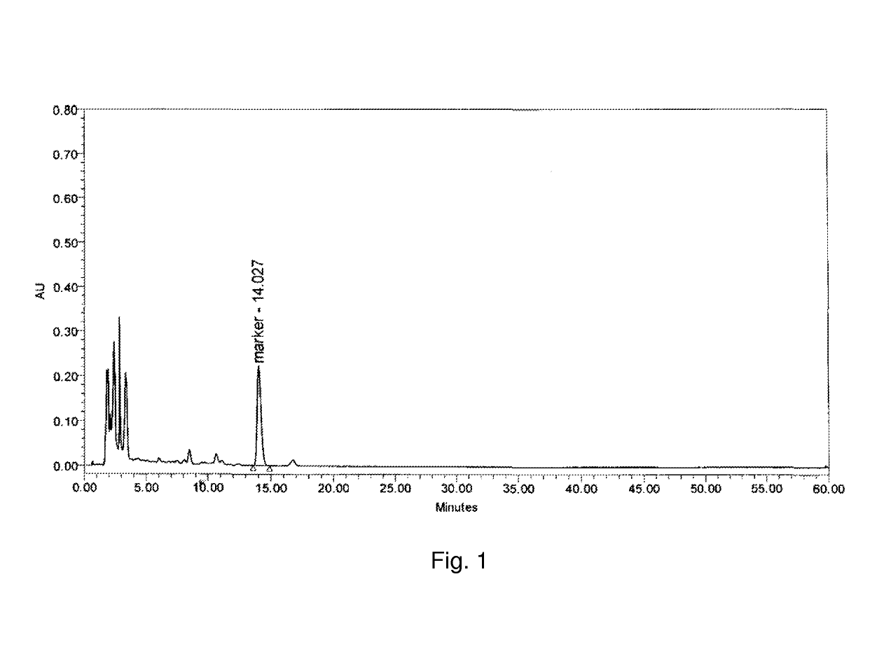

It can be shown in FIG. 1 that several peaks at around 1 to 5 minutes, together with a peak at around 14 minutes (corresponding to ergosta-7,22-dien-3(3-ol), were observed.

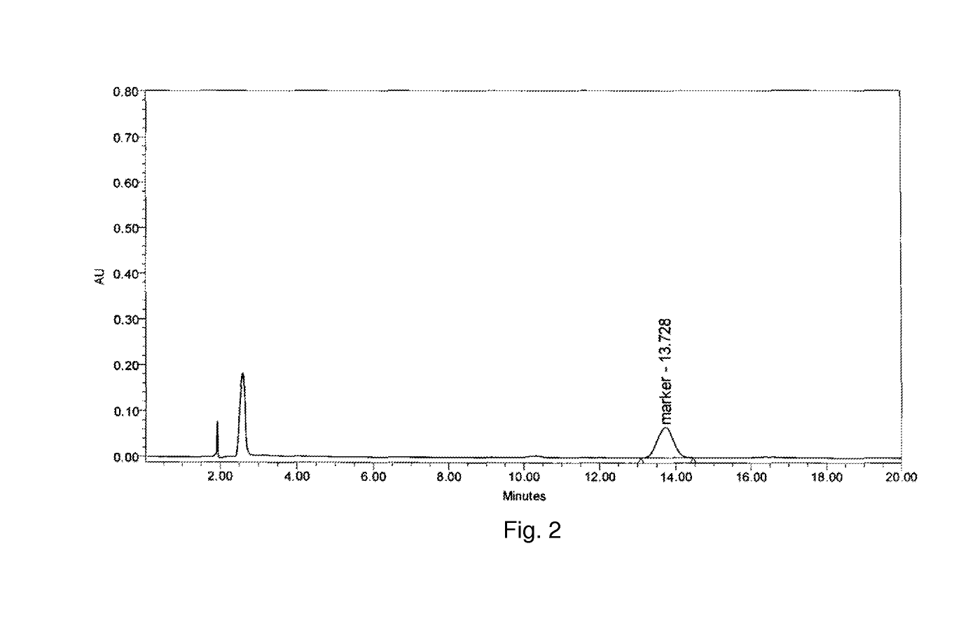

A peak at around 14 minutes was also observed in FIG. 2. This was confirmed to be the ergosta-7,22-dien-3β-ol peak and that the crystals prepared according to the method described in steps 1.1 to 1.4 were substantially pure.

The aforesaid substantially purified ergosta-7,22-dien-3β-ol crystals, when characterized with other positive identification results, possess a structure as described by formula (I) above.

1.5.2 Gas Chromatography (GC)

1.5.2.1 Preparation of Specimen

About 5 mg of the sample tested (i.e. the purified extract of ergosta-7,22-dien-3β-ol obtained from step 1.4) was dissolved with 2 mL of chloroform in a 5 mL volumetric flask. Then, chloroform was added to fill up to the mark. The solution thus formed was filtered through 0.45 μm filter into vials as specimen for GC detection.

1.5.2.2 Preparation of Samples of Impure ergosta-7,22-dien-3β-ol

About 5 mg of the ergosta-7,22-dien-3β-ol crude product obtained from step 1.3 above was dissolved with 2 mL of chloroform in a 5 mL volumetric flask. Then, chloroform was added to fill up to the mark. The solution thus formed was filtered through 0.45 μm filter into vials as specimen for GC detection.

1.5.2.3 GC System

The entire GC system and all its mechanical parts were purchased from PerkinElmer, USA. The instruments used included Clarus 600 gas chromatographic system with Flame Ionization Detector.

1.5.2.4 Running Conditions

The running conditions for this GC analysis are shown in Table 4 below.

TABLE 4

| | |
|---|---|
| Column: | PerkinElmer Elite-1 column, 15 m, |
| | 0.25 mm ID, 1 μm film thickness |
| Carrier gas and flow rate: | Nitrogen at 0.7 ml/min (constant flow) |
| Injector: | Split/splitless, 250° C. |
| Injection volume: | 1.0 μl |
| Injection: | Split with split flow at 20 ml/min |
| Detector temperature: | 300° C. |
| Oven temperature | 210° C. initially, ramp at |
| programming. | 2.5° C. per min until 275° C., |
| | and hold for 35 min |
| Total running time: | 61 min |

1.5.2.5 Results and Analysis

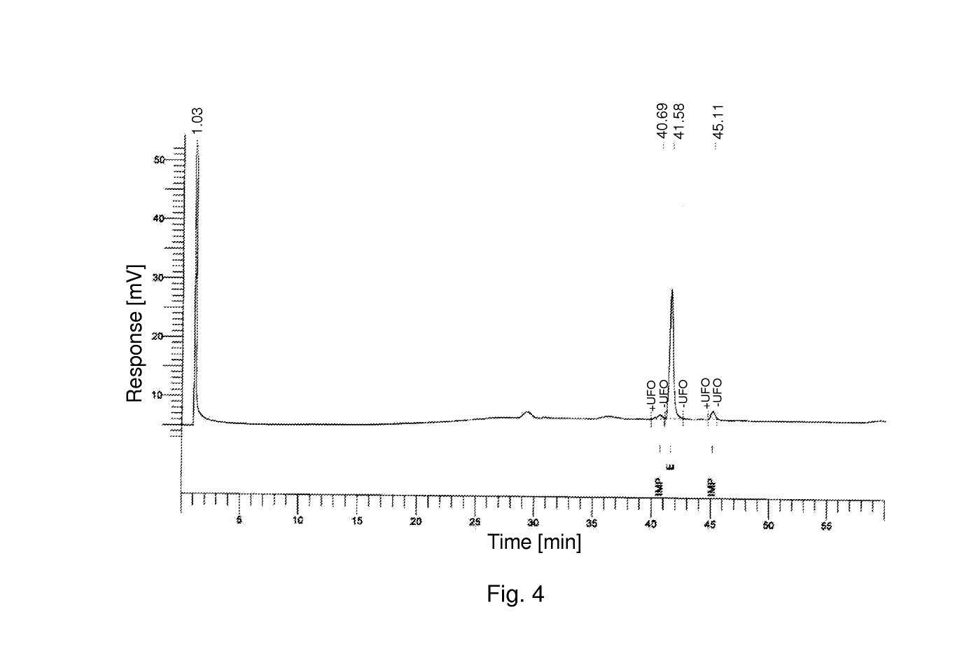

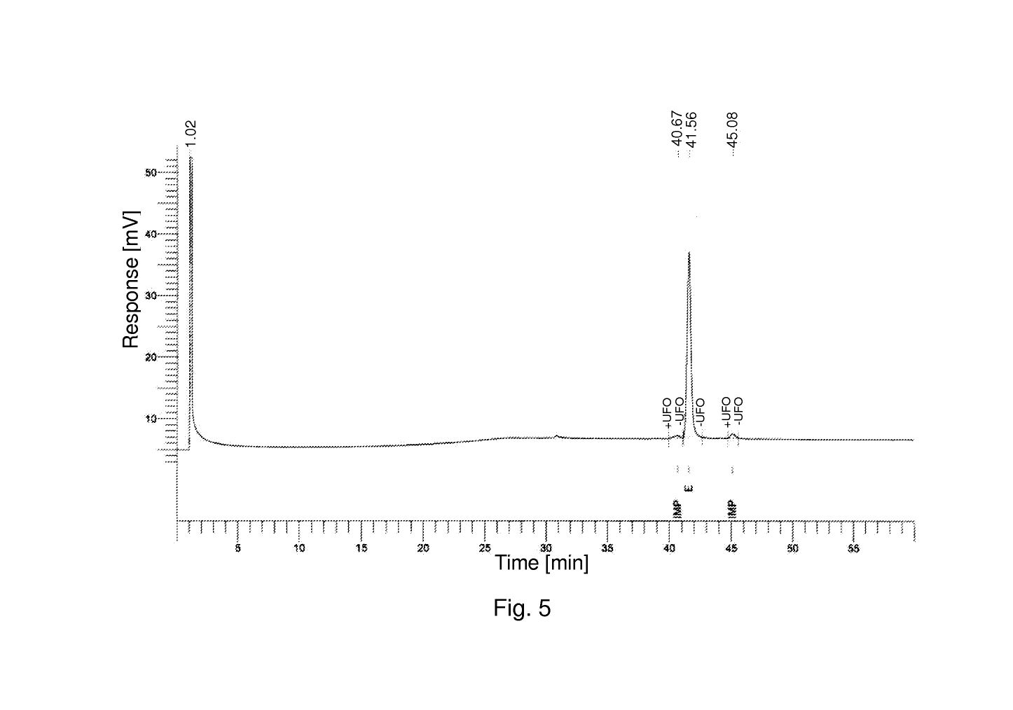

It can be shown in FIG. 4 that several peaks at around 25 to 50 minutes, together with a peak at around 41.5 minutes (corresponding to ergosta-7,22-dien-3β-ol), were observed. A peak at around 41.5 minutes was also observed in FIG. 5. This was confirmed to be the ergosta-7,22-dien-3β-ol peak and that the crystals prepared according to the method described in steps 1.1 to 1.4 were substantially pure.

The aforesaid substantially purified ergosta-7,22-dien-3β-ol crystals, when characterized with other positive identification results, possess a structure as described by formula (I) above.

### Example 2

### Use of Ergosta-7,22-Dien-3β-ol as a Marker for Coriolus Versicolor

2.1 High Performance Liquid Chromatography

2.1.1 Preparation of Specimen

Powders of ergosta-7,22-dien-3β-ol were obtained according to the method described in Example 1. The HPLC conditions used and the chromatographic results were the same as those described in Example 1. The specimen was prepared according to Section 1.5.1.1 in Example 1 above.

2.1.2 Preparation of Sample

A sample of about 500 mg Yunzhi plant was ultrasonically extracted with 40 mL of an aqueous mixture of THF-Tween 80 (in the ratio of 1:1, (THF/Tween 80): water) in a 50 mL volumetric flask for 10 minutes. Then, acetonitrile was added to fill up to the mark and the resulting solution was sonicated for another 5 minutes. The extracts thus formed were filtered through 0.45 μm filter into vials as specimen for HPLC detection.

2.1.3 HPLC System and Running Conditions

Please refer to sections of 1.5.1.3 and 1.5.1.4 in Example 1 above for detail.

2.1.4 Results and Analysis

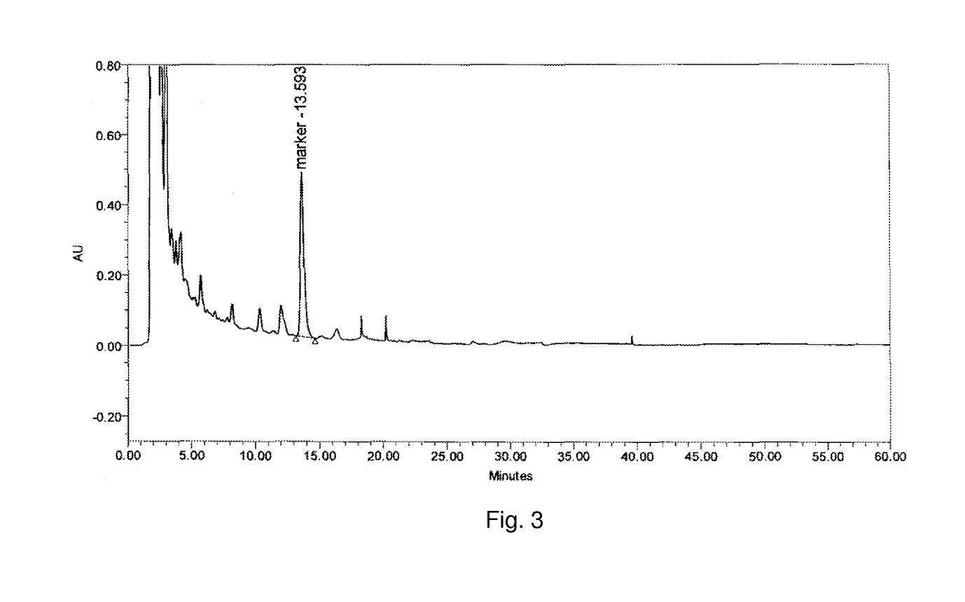

From FIG. 3, a peak at around 14 minutes (corresponding to ergosta-7,22-dien-3β-ol) was observed in the Yunzhi plant sample spectrum, indicating the presence of ergosta-7,22-dien-3β-ol in Yunzhi plant samples.

2.2 Gas Chromatography

2.2.1 Preparation of Specimen

Powders of ergosta-7,22-dien-3β-ol were obtained according to the method described in Example 1. The GC conditions used and the chromatographic results were the same as those described in Example 1. The specimen was prepared according to Section 1.5.2.1 in Example 1 above.

2.1.2 Preparation of Sample

A sample of about 2 g Yunzhi plant was ultrasonically extracted with 100 mL of a mixture of chloroform and methanol (in the ratio of 2:1, chloroform:methanol) in a 100 mL volumetric flask for 20 minutes. 50 ml of the filtered extract was dried under reduced pressure. 2 ml of chloroform and an internal standard solution (20 mg of 5α-cholestane in 2 ml of chloroform) were added to dissolve the residue. The extracts thus formed were filtered through 0.45 μm filter into vials as specimen for GC detection.

2.1.3 GC System and Running Conditions

Please refer to sections of 1.5.2.3 and 1.5.2.4 in Example 1 above for detail.

2.1.4 Results and Analysis

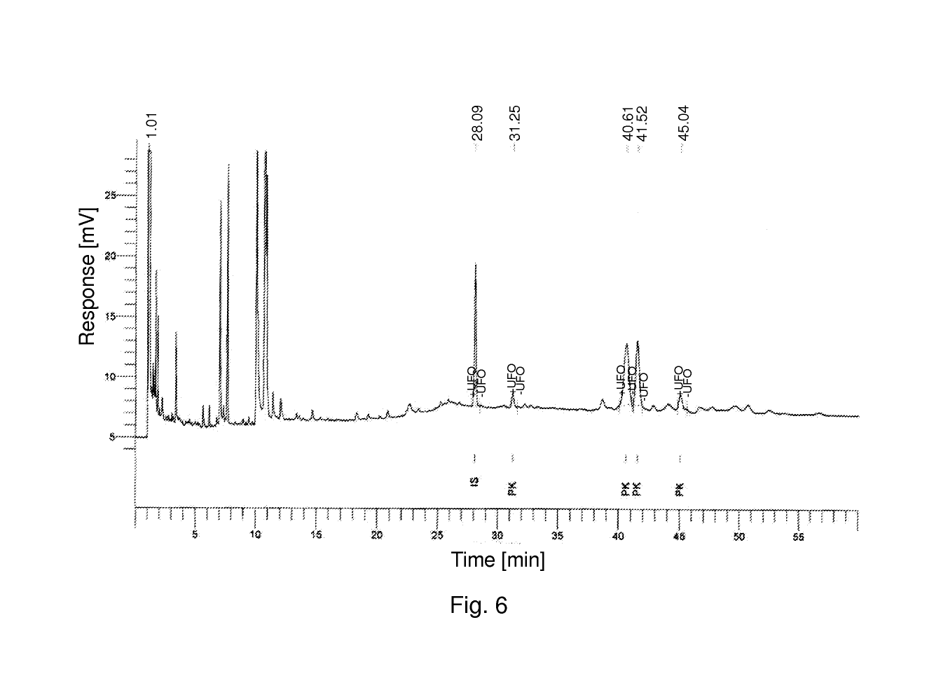

From FIG. 6, a peak at around 41.5 minutes (corresponding to ergosta-7,22-dien-3β-ol) was observed in the Yunzhi plant sample spectrum, indicating the presence of ergosta-7,22-dien-3β-ol in Yunzhi plant samples.

### Example 3

### Use of Ergosta-7,22-Dien-3β-ol for Treatment of Leukemia

The following study on nude mice was conducted to investigate the in vivo anti-tumor activities, specifically the anti-leukemia effects, of ergosta-7,22-dien-3β-ol.

3.1 Mice and Tumor Inoculation

Athymic nude mice (BALB/c nu/nu, with body weight of 20-25 g and aged 6-10 weeks) were kept in autoclaved cages with polyester fiber filters to avoid contact with the pathogens. All the animal diet and tap water were autoclaved before feeding to nude mice ad libitum.

Suspension of 1×10⁷ of human promyelocytic leukemia HL-60 cells was then injected subcutaneously (s.c.) into the dorsal side of nude mice.

3.2 Administration Schedule and Tumor Measurement

In this study, the tested item was the powders of ergosta-7,22-dien-3β-ol obtained according to the method described in Example 1 above.

When palpable tumors (about 100-200 mm³ in volume) arose within 14-21 days after injection, the mice were randomly divided into three treatment groups and one control group in which each group consisted of 15 mice. Then, the mice in the treatment group were administrated with the tested item at three dose levels of 1.5, 15.0 or 50.0 mg (of tested item)/kg (of body weight of mouse)/day (in distilled water), while those mice in the control group were administrated with distilled water (as control vehicle). Both groups of mice were administrated by oral route (gastric gavage) daily (except Sundays) for up to 28 days. Tumor dimension was measured on every Mondays, Wednesdays and Fridays.

Tumor dimension was measured by Vernier caliper and tumor weight and tumor volume were calculated according to the following formulae:

Tumor weight (mg)=L×W²×π/6; and

Tumor volume (mm³)=L×W×H×π/6;

where L, W, and H were the major dimension, the minor dimension, and the height of the tumor respectively (π=3.1416).

The relative tumor weight was calculated by dividing the tumor weight on a certain day by the mean tumor weight on Day 1. Similarly, the relative tumor volume was calculated by dividing the tumor volume on a certain day by the mean tumor volume on Day 1.

Upon 28 days of administration, the nude mice were euthanized and the final body and tumor weights were recorded for calculating the ratio of the final tumor-to-body weight.

3.3 Results and Discussion

All the three treatment groups showed some effects in the reductions in both the relative tumor weight and the relative tumor volume. Also, the percentage ratios of final tumor-to-body weight for the three treatment groups were lower than that of the control group. Among the three treatment groups, the 1.5 mg/kg/day dosage group showed the most significant results in the three aforesaid attributes.

By way of example only, the results of the percentage of inhibition of the tested item on the tested mice for the 1.5 mg/kg/day dosage group were also tabulated in Table 5 below.

TABLE 5

| | % inhibition for 1.5 mg/kg/day |
|---|---|
| Day (n) | dosage group |
| 1 | 0.00 |
| 3 | 11.35 |
| 5 | 11.24 |
| 8 | 23.69 |
| 10 | 31.65 |
| 12 | 32.32 |
| 15 | 30.61 |
| 17 | 34.99 |
| 19 | 43.19 |
| 22 | 38.94 |
| 24 | 40.57 |
| 26 | 44.32 |

The above-shown results were expressed as the mean±SE of the percentage of inhibition of the tested item on the growth of HL-60 xenografts in nude mice in single experiments. Percentage of inhibition (for any Day n) was calculated from the following formula:

(1−relative tumor weight of treatment group on Day n/relative tumor weight of control group on Day n)×100%.

From Table 5, the 1.5 mg/kg/day dosage group was demonstrated to show inhibition on HL-60 xenograft, with the percentage thereof being up to maximum 44.32% on Day 26.

3.4 Conclusion

Anti-tumor activity of ergosta-7,22-dien-3β-ol was observed at all three dosage groups. Doses that may be used include but are not limited to 1.0 to 20.0 mg/kg body weight/day. In a non-limiting embodiment, dosages that may be used include but are not limited to 1.0 to 5.0 mg/kg body weight/day.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, GC and HPLC are mentioned to separate the sample extract in the method of identifying and/or quantifying Yunzhi, but it is clear to one skilled in the art that other separation techniques such as other chromatographic techniques (such as liquid or thin layer chromatography), spectroscopy, fractional distillation and liquid-liquid extraction may also be used.

Further, the method of quantifying Yunzhi using ergosta-7,22-dien-3β-ol can be employed in different stages during the preparation of Yunzhi extract or Yunzhi formulation. For example, in the preparation of a Yunzhi formulation, the Yunzhi starting material can first be quantified using the aforesaid method. Then, upon extraction, the extract can also be tested with this method. Lastly, the final product of Yunzhi formulation resulted from the mixing of the extract and other components can also be quantified with this method.

What is claimed is:

1. A method of treating cancer comprising administrating an effective amount of a formulation comprising isolated and purified ergosta-7,22-dien-3β-ol.

2. The method according to claim 1 wherein said isolated and purified ergosta-7,22-dien-3β-ol has a gas chromatography fingerprint as shown in FIG. 5.

3. The method according to claim 1 wherein said cancer is leukemia.

4. The method according to claim 1 wherein said formulation further comprises said ergosta-7,22-dien-3β-ol admixed with other herb(s) and/or other pharmaceutically acceptable carrier(s).

5. A method of treating cancer comprising administrating an effective amount of a formulation consisting of isolated and purified ergosta-7,22-dien-3β-ol.

6. The method according to claim 5 wherein said isolated and purified ergosta-7,22-dien-3β-ol has a gas chromatography fingerprint as shown in FIG. 5.

7. The method according to claim 5 wherein said cancer is leukemia.
